(19) United States
(12) Patent Application Publication
(10) Pub. No.: US 2021/0402397 A1
(43) Pub. Date: Dec. 30, 2021

(54) BIDIRECTIONAL FLUID FLOW IN A MICROFLUIDIC DEVICE

(71) Applicant: Singular Genomics Systems, Inc., La Jolla, CA (US)

(72) Inventors: Tuan Tran, San Diego, CA (US); Terrence Murphy, Oceanside, CA (US); Eli N. Glezer, Del Mar, CA (US)

(21) Appl. No.: 17/354,798

(22) Filed: Jun. 22, 2021

Related U.S. Application Data
(60) Provisional application No. 63/044,859, filed on Jun. 26, 2020.

(51) Int. Cl. B01L 3/00; C12Q 1/6869

(52) U.S. Cl. CPC B01L 3/502715; B01L 3/52; B01L 2300/0877; B01L 2200/141; B01L 2200/10; C12Q 1/6869

(57) ABSTRACT

Provided herein, inter alia, are nucleic acid sequencing devices and flow cells containing different flow paths to control the flow of fluidic solutions.

### CROSS-REFERENCES TO RELATED APPLICATIONS

This application claims the benefit of U.S. Provisional Application No. 63/044,859, filed Jun. 26, 2020, which is incorporated herein by reference in its entirety and for all purposes.

### BACKGROUND

Manipulating fluidic reagents and assessing the results of reagent interactions are central to chemical and biological science. In particular, fluidic manipulations within a nucleic acid sequencing instrument are complex and, if performed incorrectly, can negatively impact the sequencing results. Many of the next-generation sequencing (NGS) technologies use a form of sequencing by synthesis (SBS), wherein modified nucleotides are used along with an enzyme to read the sequence of DNA templates in a controlled manner. Other NGS platforms use native nucleotides or labeled oligonucleotides with ligation enzymes to determine nucleic acid sequences. NGS technologies demand a robust and efficient fluidic architecture, capable of delivering precise volumes of solutions (i.e., reagents) for hundreds, and in many cases, thousands, of fluidic exchange cycles for a single experiment.

### SUMMARY

Disclosed herein, inter alia, are solutions to these and other problems in the art. For example, described herein is a microfluidic device comprising a flow cell which is fluidically connected by at least two different flow paths. This results in greater efficiency and functionality by allowing two or more independent reactions, such as a sequencing reaction and amplification reaction, to occur on the same, common or single flow cell (i.e., the reaction chamber) without cross-contamination and allows for a reduction in the overall path length on each side of the flow cell, thereby saving critical reagent volume.

In one aspect, there is disclosed a flow cell system (e.g., a device or apparatus, such as a microfluidic or nucleic acid sequencing device), comprising: at least one flow cell (e.g., one or two flow cells) configured to serve as a reaction vessel in a nucleic acid sequencing device, the flow cell including at least one fluidic channel through which a fluid solution can flow; an inlet to the at least one fluidic channel; an outlet to the at least one fluidic channel; wherein two or more independent reactions can occur (e.g., simultaneously occur) on the at least one flow cell with minimal or a reduction in cross-contamination. In embodiments, two or more independent reactions can occur in the same or common channel of the flow cell with at least two or more independent reactions occurring at different points in time. In embodiments, two or more independent reactions can occur in different channels of the flow cell at the same or different points in time.

In another aspect, there is disclosed a method of performing a reaction on a flow cell, comprising: performing at least two independent reactions on a common flow cell with minimal or reduced cross-contamination. In embodiments, each channel is individually addressable (i.e., each channel within the flow cell is capable of performing an independent, optionally different, experiment). For example, in a four-channel flow cell, while one or more sequencing reactions are occurring in one channel, one or more amplification reactions may be occurring simultaneously in the remaining channels. In embodiments, each channel of the flow cell is capable of performing the same reaction simultaneously, optionally under different conditions. For example, in a four-channel flow cell, one channel may sequence under a particular set of conditions (e.g., performing sequencing reactions in the presence of a buffer containing 1 mM NaCl), and the remaining channels may sequence under different conditions (e.g., performing sequencing reactions in the presence of a buffer containing 0.5 mM NaCl).

The microfluidic device and fluidic subsystems described herein are applicable for amplifying (i.e., clustering), processing, and/or detecting samples of analytes of interest in a flow cell. Within this application the fluidic system is made in reference to nucleic acid sequencing (i.e., a genomic instrument) which allows for the sequencing of nucleic acid molecules. However, the techniques disclosed herein may be applied to any system making use of reaction vessels, such as flow cells, for detection of analytes of interest, and into which solutions are introduced during preparation, reaction, detection, or any other process on or within the reaction vessel. The systems and methods described herein are useful for performing at least two independent reaction modes (e.g., amplification and sequencing) in the same channel of the flow cell at different times.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIGS. 1A-1B show example embodiments of a flow cell wherein two different solutions are configured to flow from an inlet to an outlet via one or more channels. The channels are not explicitly depicted in the illustration although the channels are present in the flow cell.

FIGS. 2A-2B show example embodiments of a bi-directional flow cell configured to support reciprocating flow.

FIGS. 3A-3D show example embodiments of a flow cell coupled to fluid flow manifold.

### DETAILED DESCRIPTION

Flow cells provide a convenient format for housing an array that is produced by the methods of the present disclosure and that is subjected to a sequencing-by-synthesis (SBS) or other detection technique that involves repeated delivery of reagents in cycles. A flow cell may include a patterned array, such as a microarray or a nanoarray. The locations or sites may be disposed in a regular, repeating pattern, a complex non-repeating pattern, or in a random arrangement on one or more surfaces of a support. To enable the sequencing chemistry to occur, the flow cell also allows for introduction of fluidic solutions, such as reagents, buffers, nucleotides, enzymes, and other substances involved in the reactions, as well as solutions used for flushing or cleaning the fluidic manifolds or flow cell. The solutions flow through the flow cell and may contact the molecules of interest at the individual sites.

One reaction that may occur within the flow cell is an amplification reaction, alternatively referred to herein as clustering. A nucleic acid can be amplified by any suitable method known in the art. The term “amplified” and “amplification” refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. Typically, non-terminated nucleotides are used in amplification reaction. Conditions conducive to amplification (i.e., amplification conditions) are well known and often comprise at least a suitable polymerase, a suitable template, a suitable primer or set of primers, suitable nucleotides (e.g., non-terminated dNTPs), a suitable buffer, and application of suitable annealing, hybridization and/or extension times and temperatures.

It will be appreciated that any of the amplification methodologies described herein or known in the art can be utilized with universal or target-specific primers to amplify (i.e., generate clusters of) the target polynucleotide. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. Additional examples of amplification processes include, but are not limited to, bridge-PCR, recombinase polymerase amplification (RPA), loop-mediated isothermal amplification (LAMP), rolling circle amplification (RCA), strand displacement amplification, RCA with exponential strand displacement amplification. In embodiments, amplification comprises an isothermal amplification reaction. In embodiments, amplification comprises bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site. Examples of additional clonal amplification techniques include, but are not limited to, bridge PCR, solid-phase rolling circle amplification (RCA), solid-phase exponential rolling circle amplification, solid-phase recombinase polymerase amplification (RPA), solid-phase helicase dependent amplification (HDA), template walking amplification, emulsion PCR on particles (beads), or combinations of the aforementioned methods. Optionally, during clonal amplification, additional solution-phase primers can be supplemented in the microplate for enabling or accelerating amplification. In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, Pol I DNA polymerase, SD polymerase, Bst large fragment polymerase, or a phi29 polymerase or mutant thereof.

In embodiments, amplifying includes contacting the flow cell with one or more reagents (i.e., a clustering solution) for amplifying the target polynucleotide. Examples of reagents include but are not limited to polymerase, buffer, and nucleotides (e.g., an amplification reaction mixture). In certain embodiments the term “amplifying” refers to a method that includes a polymerase chain reaction (PCR). Conditions conducive to amplification (i.e., amplification conditions) are known and often comprise at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, amplifying includes a bridge polymerase chain reaction amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and one or more additives (e.g., ethylene glycol) and maintaining the temperature within a narrow temperature range (e.g., +/−5° C.) or isothermally. In embodiments, c-bPCR does not include isothermal amplification, rather it requires minor (e.g., +/−5° C.) thermal oscillations. In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, amplifying includes generating a double-stranded amplification product.

Another such reaction that may occur within the flow cell is a sequencing reaction. In a sequencing reaction, cyclic operations are implemented in an automated or semi-automated manner to promote nucleic acid incorporation and detection. In SBS, the use of labeled nucleotides bearing a 3′ reversible terminator (RT) allows successive nucleotides to be incorporated into a polynucleotide chain in a controlled manner. The DNA template for a sequencing reaction will typically comprise a double-stranded region having a free 3′ hydroxyl group which serves as a primer or initiation point for the addition of further nucleotides in the sequencing reaction. The region of the DNA template to be sequenced will overhang this free 3′ hydroxyl group on the complementary strand. The primer bearing the free 3′ hydroxyl group may be added as a separate component (e.g. a short oligonucleotide) which hybridizes to a region of the template to be sequenced. Following the addition of a single nucleotide to the DNA template, the presence of the 3′ reversible terminator prevents incorporation of a further nucleotide into the polynucleotide chain. While the addition of subsequent nucleotides is prevented, the identity of the incorporated is detected (e.g., exciting a unique detectable label that is linked to the incorporated nucleotide). The reversible terminator is then removed, leaving a free 3′ hydroxyl group for addition of the next nucleotide. The sequencing cycle can then continue with the incorporation of the next blocked, labeled nucleotide. A sequencing cycle may include introducing modified nucleotides (e.g., labeled or non-labeled nucleotides with a reversible terminator) and enzymes, followed by flushing with a wash solution.

The flow cell as described herein includes one or more fluidic channels that allow for two or more independent reactions, such as amplification and/or sequencing chemistry to occur within the same reaction chamber, at different time points. As described above, nucleic acid amplification reactions utilize different solutions than sequencing reactions. In particular, nucleic acid amplification reactions typically occur in the presence of non-terminated nucleotides (i.e., native nucleotides). It is crucial to keep the two different solutions distinct and separate between the different reactions (i.e., the sequencing reaction and the amplification reaction). Allowing cross-contamination of solutions used in an amplification reaction with sequencing solutions, and vice versa, will negatively impact the sequencing quality.

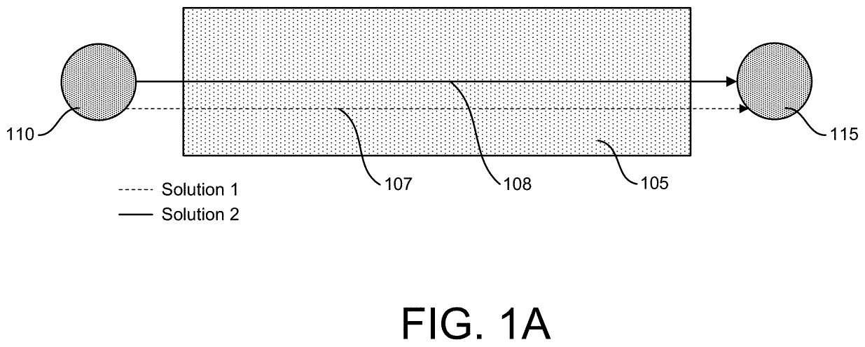

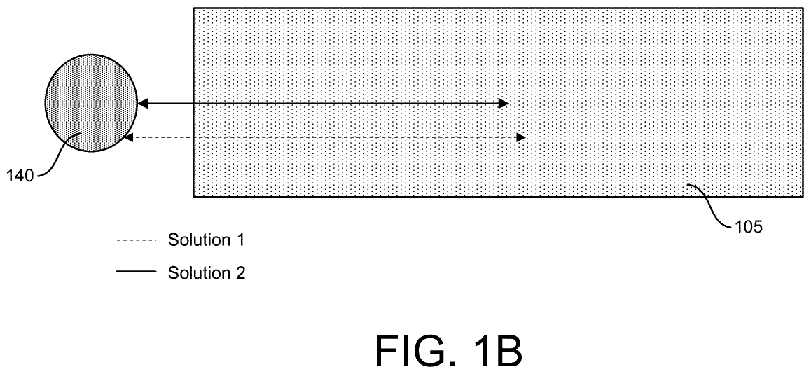

Fluidic flow through a microfluidic device typically proceeds unidirectionally. For example, a simplified diagram showing cyclical flow is depicted in FIG. 1A, which shows a flow cell 105 having two flow channels 107 and 108 that provide fluid communication between an inlet 110 and an outlet 115. It should be appreciated that the quantity of channels of the flow cell 105 can vary. For example, the quantity can be one, two, or more channels. In this case, all reagents (i.e., solutions) flow into channel through the inlet 110, and out of the channel through the outlet 115. A solution 1 and a solution 2 flow from the inlet 110 to the outlet 115 via a respective channel. The solution(s) can be, for example, a reagent. FIG. 1A shows the reagents flow into a respective channel through the inlet 110 and out of the respective channel through the outlet 115.

In any of the embodiments described herein, each channel has a structure configuration that is configured to enable fluid flow through a channel. A structure configuration of at least one of the channels can vary. In an embodiment, at least one channel has a cross sectional shape of a circle, rectangle, oval, or any other shape. Preferably, the flow rates, fluid viscosities, compositions, and geometries and sizes of the channel are selected so that fluid flow is laminar. Guidance for configuring such channel is readily available publicly available resources, for example Acheson, Elementary Fluid Dynamics (Clarendon Press, 1990), and from software for modeling fluidics systems, e.g. SolidWorks from Dassault Systems. In an embodiment, at least one channel has passage cross-sections in the range of tens of square microns to a few square millimeters (e.g., maximal cross-sectional dimensions of from about 500 μm to about 0.1 μm). In an embodiment, the flow rates in the range of from a few nL/sec to a hundreds of μL/sec. In an embodiment, volume capacities in are the range of from 1 μm to a few nL, e.g. 10-100 nL.

Alternatively, the inlet can be used as an outlet, as depicted in FIG. 1B, which shows an alternate embodiment wherein a single component or structure 140 serves as both an inlet and as an outlet for one or more channels. In this embodiment, reciprocating flow is achieved in that the two different solutions enter and exit the channel through the same or common structure 140. However, preventing cross-contamination of the two different solutions may limit the applicability of such an orientation. Optionally, the reagents may be recycled. The system may optionally include a recycling reservoir.

Achieving bidirectional flow in a microfluidic device results in greater functionality, allowing different independent reactions to occur within the same flowcell (e.g., sequencing and clustering (i.e., generating a plurality of polynucleotides) on the same flowcell) without cross-contamination. Additionally, the design reduces the overall path length on each side of the flowcell, thereby saving reagent volume. The direction and rate of flow through junctions, nodes and passages of the fluidics circuit are controlled by the states of valves (e.g., opened or closed), differential fluid pressures at circuit inlets or upstream reservoirs, flow path resistances, and the like.

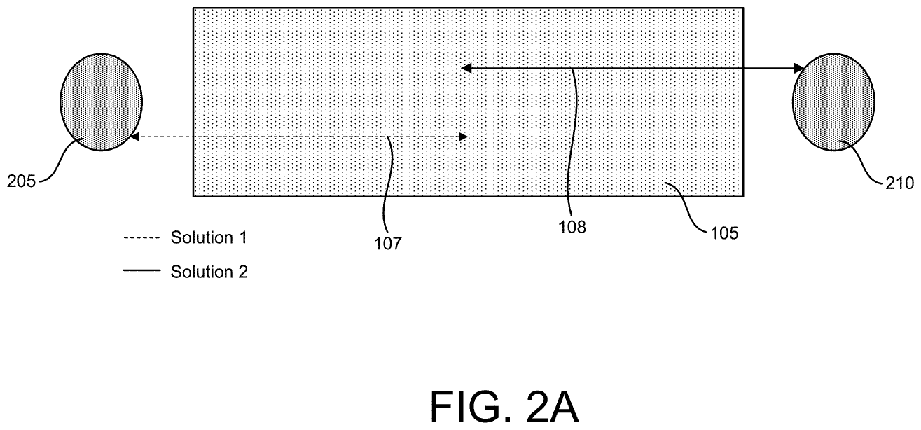

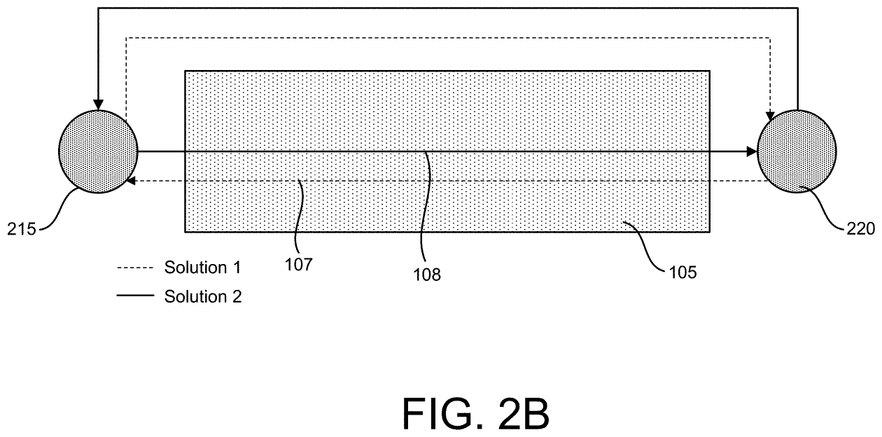

Depicted in FIG. 2A is an embodiment of a bidirectional flow cell with reciprocating flow, where the inlet serves as both the inlet and the outlet. FIG. 2A thus shows an embodiment of a bidirectional flow cell 105 configured to support reciprocating flow. A structure 205 of the flow cell 105 serves as both an inlet and an outlet (i.e., where a solution can enter and exit the channel through the same inlet structure) for a first solution (solution 1) via a respective channel. A structure 210 of the flow cell 105 serves as both an inlet and an outlet for a second solution (solution 2) via a respective channel. It should be appreciated that the quantity of channels of the flow cell 105 can vary and can be one, two, or more channels.

Alternatively, cyclical flow can be achieved within a bidirectional flow cell 105. FIG. 2B shows another embodiment of a bidirectional flow cell 105 configure to achieve cyclical flow within a bidirectional flow cell. An inlet 215 for a first solution acts as an outlet for a second solution. Similarly, the inlet 220 for the second solution also acts as an outlet for the first solution. The first solution flows through a flow channel 107 and the second solution flows through a flow channel 108. Thus, in this embodiment, a first solution's inlet serves as the outlet for another solution and the second solution's inlet serves as an outlet for another solution.

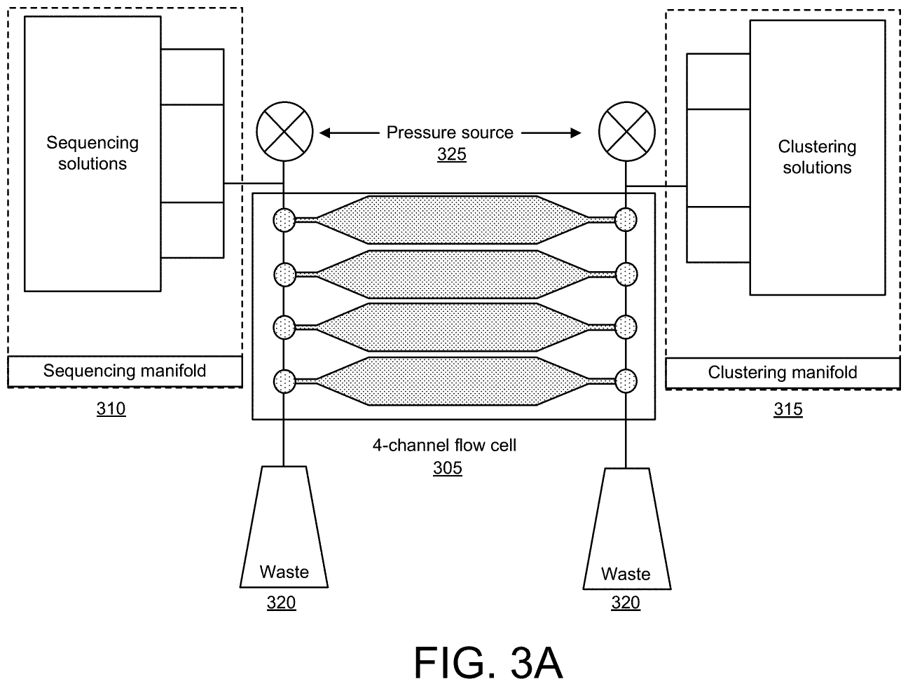

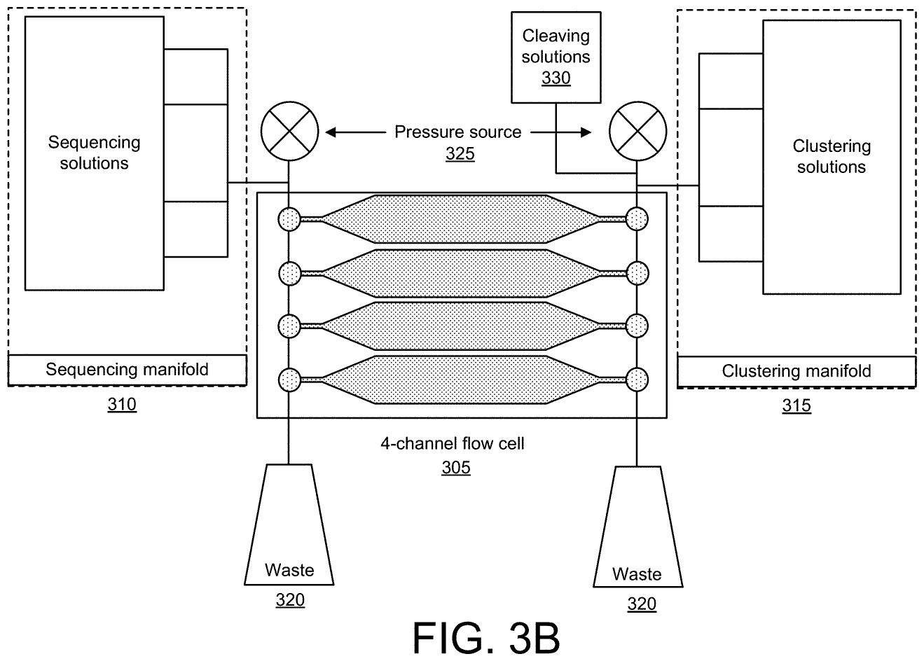

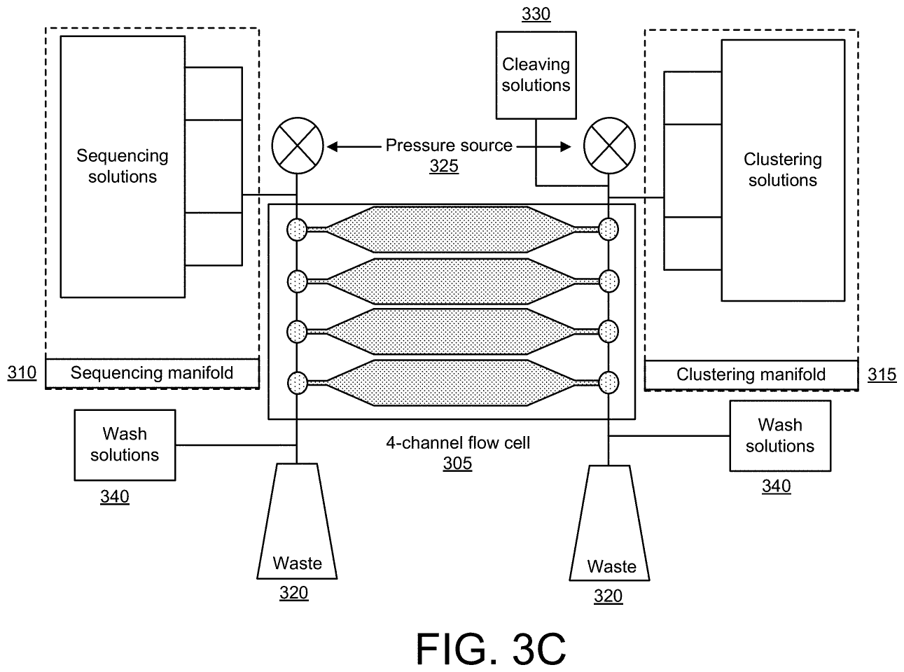

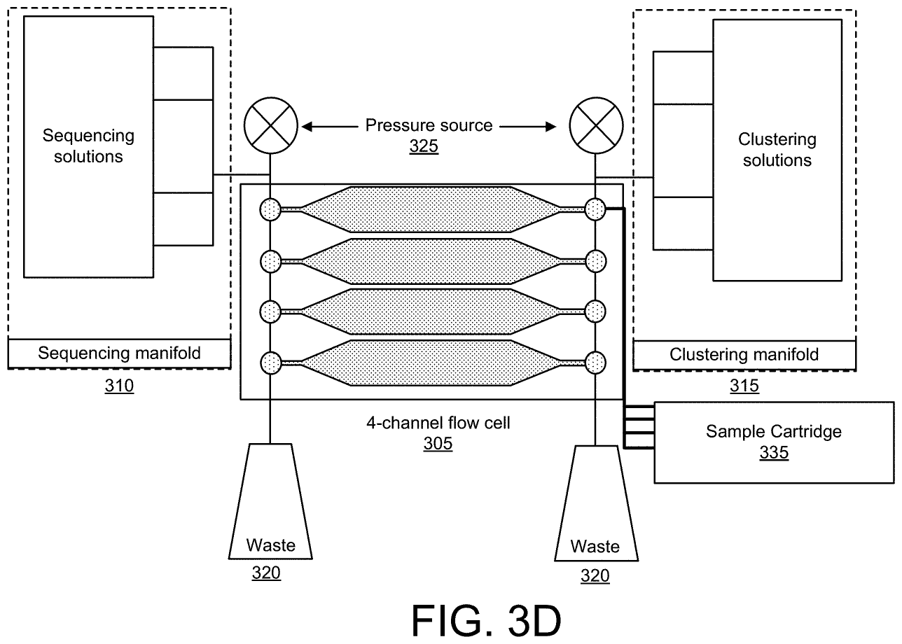

In embodiments, the sequencing solutions (e.g., terminated nucleotides) are delivered from one side of the flow cell, and clustering solutions (e.g., amplification solution containing non-terminated nucleotides (i.e., native nucleotides)) are delivered from the other side (such as an opposite side) of the flow cell. The embodiments of FIGS. 3A-3D show a version where the flow cell 305 is a four-channel, bidirectional flow cell although the quantity of channels can vary. In the embodiment of FIG. 3A, a manifold 310 is positioned on a first side of the flow cell 305. The manifold 310 comprises a fluidic pipe or chamber that branches into two or more fluidic passageways. The manifold 310 is configured to deliver sequencing solutions (e.g., terminated nucleotides), while a second manifold 315 is configured to deliver clustering solutions (e.g., non-terminated nucleotides) from the other side of the flow cell.

In embodiments, the sequencing solution includes (a) an adenine nucleotide, or analog thereof; (b) (i) a thymine nucleotide, or analog thereof, or (ii) a uracil nucleotide, or analog thereof; (c) a cytosine nucleotide, or analog thereof; and (d) a guanine nucleotide, or analog thereof. In embodiments, the sequencing solution includes a plurality of adenine nucleotides, or analogs thereof; a plurality of thymine nucleotides, or analogs thereof, or a plurality of uracil nucleotides, or analogs thereof; a plurality of cytosine nucleotides, or analogs thereof; and a plurality of guanine nucleotides, or analogs thereof. In embodiments, each sequencing cycle includes contacting the complementary polynucleotide with a sequencing solution, wherein the sequencing solution comprises one or more nucleotides, wherein each nucleotide comprises a reversible terminator. In embodiments, each sequencing cycle includes contacting the complementary polynucleotide with a sequencing solution, wherein the sequencing solution comprises one or more nucleotides, wherein each nucleotide comprises a reversible terminator and a label. In embodiments, the sequencing solution includes a plurality of nucleotides, each nucleotide including a 3′-reversible terminator and a detectable label. For example, a nucleotide including a reversible terminator moiety may be represented by the formula:

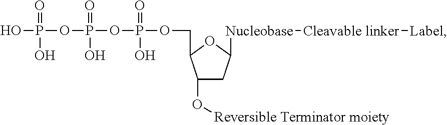

where the nucleobase is adenine or adenine analogue, thymine or thymine analogue, guanine or guanine analogue, or cytosine or cytosine analogue. In embodiments, the detectable label is a fluorescent dye. In embodiments, sequencing a template nucleic acid includes extending a complementary polynucleotide that is hybridized to the template nucleic acid by incorporating a first nucleotide. In embodiments, the nucleotide is selected from one or more of dATP, dCTP, dGTP, and dTTP or an analogue thereof. In embodiments, the nucleotide includes a detectable label. In embodiments, the detectable label is a fluorescent label. In embodiments, the nucleotide includes a reversible terminator moiety. In embodiments, the reversible terminator moiety may be 3′-O-blocked reversible terminator. In nucleotides with 3′-O-blocked reversible terminators, the blocking group (referred to as —OR) wherein the O of —OR is the oxygen atom of the 3′-OH of the pentose, and R of —OR is the blocking group (i.e. the reversible terminator moiety) while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the method comprises a plurality of cycles, with each cycle comprising incorporation and identification of a first nucleotide. In some embodiments of methods comprising a plurality of sequencing cycles, the first nucleotide incorporated in one cycle of the plurality of cycles may be the same or different from the first nucleotide incorporated in another cycle of the plurality of cycles.

In embodiments, the sequencing solution includes a plurality of modified nucleotides. In embodiments, the nucleotides in the sequencing solution have the formula:

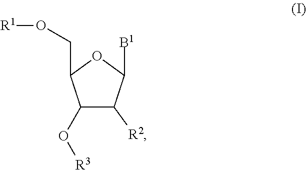

wherein B¹ is a nucleobase (e.g., a nucleobase including a covalent linker optionally bonded to a detectable moiety, for example as described herein). In embodiments, B′ is a substituted or unsubstituted nucleobase (e.g., —B-L¹⁰⁰-R⁴); R¹ is —OH, a monophosphate moiety, or polyphosphate moiety (e.g., triphosphate); R² is —OH or hydrogen; and R³ is a reversible terminator moiety. In embodiments, R² is hydrogen. In embodiments, B¹ is —B-L¹⁰⁰-R⁴; wherein B is a divalent nucleobase, L¹⁰⁰ is a divalent linker, and R⁴ is a detectable moiety (e.g., a label). In embodiments, B is a divalent cytosine or a derivative thereof, divalent guanine or a derivative thereof, divalent adenine or a derivative thereof, divalent thymine or a derivative thereof, divalent uracil or a derivative thereof, divalent hypoxanthine or a derivative thereof, divalent xanthine or a derivative thereof, divalent 7-methylguanine or a derivative thereof, divalent 5,6-dihydrouracil or a derivative thereof, divalent 5-methylcytosine or a derivative thereof, or divalent 5-hydroxymethylcytosine or a derivative thereof. L¹⁰⁰ is a divalent, cleavable, linker; and R⁴ is a detectable moiety. In embodiments, is independently a bioconjugate linker, a cleavable linker, or a self-immolative linker. In embodiments, B¹ is a divalent nucleobase. In embodiments, B¹ is

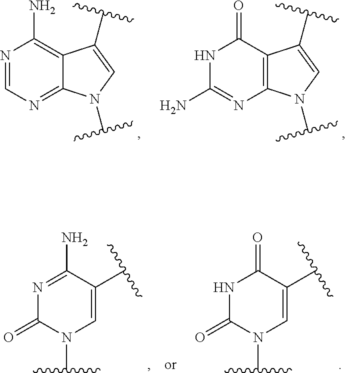 [SMILES: *c1cn(*)c2ncnc(N)c12 ; *c1cn(*)c2nc(N)[nH]c(=O)c12 ; *c1cn(*)c(=O)nc1N ; *c1cn(*)c(=O)[nH]c1=O]

In embodiments, the clustering solution includes the necessary components for amplifying and generating a plurality of polynucleotides in the flow cell. Alternatively, a clustering solution may be referred to herein as an amplification solution. For example, in embodiments, nucleotides used in the clustering solution have the formula:

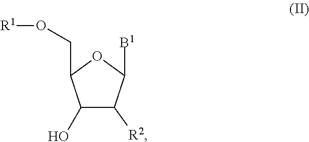

wherein R¹, R², and B¹ are as described herein, including embodiments. In embodiments, the clustering solution includes a plurality of native nucleotides, salts, ions, buffers, and enzymes.

In embodiments, the flow cell includes an array of sites on a solid-phase substrate, each site containing immobilized primers. In embodiments, each site includes a polymer-coated bead (e.g., a nanoparticle), wherein the polymer includes one or more immobilized primers. The terms “particle” and “bead” are used interchangeably and mean a small body made of a rigid or semi-rigid material. The body can have a shape characterized, for example, as a sphere, oval, microsphere, or other recognized particle shape whether having regular or irregular dimensions. A “nanoparticle,” as used herein, is a particle wherein the longest diameter is less than or equal to 1000 nanometers. Nanoparticles may be composed of any appropriate material. For example, nanoparticle cores may include appropriate metals and metal oxides thereof (e.g., a metal nanoparticle core), carbon (e.g., an organic nanoparticle core) silicon and oxides thereof (e.g., a silicon nanoparticle core) or boron and oxides thereof (e.g., a boron nanoparticle core), or mixtures thereof. Nanoparticles may be composed of at least two distinct materials, one material (e.g., silica) forms the core and the other material forms the shell (e.g., copolymer) surrounding the core. In embodiments, the flow cell includes a solid support including a surface, the surface including a plurality of wells separated from each other by interstitial regions on the surface, wherein one or more wells includes a particle, wherein the particle a plurality of oligonucleotide moieties (e.g., primers). In embodiments, there is at least one particle per well. In embodiments, there is at most one particle per well.

With reference still to FIG. 3A, the flow cell 305 is coupled to one or more pressure sources 325, which can be a pump or system of pumps configured to pull solutions with a negative pressure and to also push solutions back by inverting the pressure. This provides significant solution conservation. The pressure sources 325 can be coupled to each channel of the flow cell 305.

FIG. 3B shows an embodiment of a flow cell 305 wherein the sequencing solutions (e.g., terminated nucleotides) are delivered from one side of the flow cell 305, and cleaving solutions (e.g., solutions which remove the reversible terminator moiety), and the clustering solutions (e.g., non-terminated nucleotides) are delivered from the other, opposite side of the flow cell. The cleaving solution may be accessed from a reservoir 330 fluidly coupled to the flow cell 305. The system can include a sequencing manifold 310 and a clustering manifold 315. Separation of the delivery points of sequencing solutions and cleaving solutions (as in the embodiment of FIG. 3B) reduces the occurrence of removing the reversible terminator moiety prematurely, i.e., prior to incorporation and detection.

In the embodiment of FIG. 3C, external wash solutions 340 are coupled to the flow cell 305 outside of the sequencing manifold 310 and the clustering manifold 315. Any of the embodiments can also include waste receptacles 320 that are coupled to the channels of the flow cell.

FIG. 3D shows an embodiment of a flow cell 305 wherein the sequencing solutions (e.g., terminated nucleotides) are delivered from one side of the flow cell 305, and the clustering solutions (e.g., non-terminated nucleotides) are delivered from the other, opposite side of the flow cell. In this embodiment, the cleaving solutions (e.g., solutions which remove the reversible terminator moiety) are included in the sequencing manifold. The system can include a sequencing manifold 310 and a clustering manifold 315, each of which is fluidly coupled to the channels of the flow cell 305. Separation of the delivery points of sequencing solutions and cleaving solutions (as in the embodiment of FIG. 3B) reduces the occurrence of removing the reversible terminator moiety prematurely, i.e., prior to incorporation and detection. The sample cartridge 335 includes the input sample solution (e.g., target polynucleotides) and contains one or more reservoirs, each containing a sample of polynucleotides for sequencing. Each sample reservoir is fluidly coupled to each channel in the flow cell 305.

To initiate a sequencing experiment, the sequencing manifold selects the reagent(s) to be pulled through the flow cell, while the clustering manifold applies vacuum (i.e., negative pressure) to pull the one or more reagent(s) across flow cell. Conversely, to initiate a clustering experiment the clustering manifold selects the reagent(s) to be pulled through the flow cell while the sequencing manifold applies a vacuum to pull the one or more clustering solutions through the flow cell. The manifolds are additionally capable of bypass operation, wherein one of the manifolds (e.g., the sequencing manifold) can pull fluid by selecting a reagent and turning on its own vacuum valve. This bypasses the need to flow through the flow cell, which is useful for priming and washing the fluidics.

While a single 4-channel flow cell is illustrated in FIGS. 3A-3C, in some devices more than one flow cell and/or fluidics path may be accommodated. For example, a single 6-channel flow cell may be used, or two 4-channel flow cells. Increasing flow cells or fluidic paths enhances sequencing and throughput. In practice, any number of flow cells and paths may be provided. These may make use of the same or different reagent receptacles, disposal receptacles, control systems, and image analysis systems. The multiple fluidics systems may be individually controlled or controlled in a coordinated fashion.

One or more liquids or solutions may be degassed to improve performance of the microfluidic device and/or sequencing results. In embodiments, one or more of the solutions may be degassed upstream of the flow cell. In embodiments where more than one solution is degassed, these may be grouped in a single vacuum chamber. In embodiments where more than one solution is degassed, more than one vacuum chamber or vacuum system may be used. At any point within the flow cell, or in particular at the inlet and outlet of the flow cell, bubbles may nucleate or become lodged. The bubbles may have an adverse effect on detecting, imaging, image processing, or other operations. It is known that the number and frequency of occurrence of bubbles are reduced by degassing solutions prior to their entry into the flow cell. When any nucleated bubbles remain within the flow cell or within the fluid path, flushing a solution bidirectionally through the flow cell may aid in dislodging and removing bubbles.

In embodiments, the at least one fluid channel further includes a fluidic connection to a waste reservoir. A waste reservoir is a container capable of receiving fluids from the flow cell and/or retaining the fluidic discharge until disposing the fluids. In embodiments, the waste reservoir is capable of containing 1 L to 10 L of fluid. In embodiments, the waste reservoir is capable of containing 3 L to 6 L of fluid. In embodiments, the waste reservoir is capable of containing 5 L of fluid.

In another aspect is provided a method of amplifying and sequencing a target polynucleotide in a sequencing device comprising a flow cells system as described herein. In embodiments, the method includes a) contacting the flow cell with a target polynucleotide and amplifying the target polynucleotide to generate a plurality of immobilized template nucleic acids, wherein each immobilized template nucleic acid comprises the target polynucleotide or a complement thereof; and b) sequencing the plurality of immobilized template nucleic acids; thereby amplifying and sequencing a target polynucleotide in a sequencing device. In embodiments, amplifying includes flowing a clustering solution into one or more fluidic channels of the flow cell. In embodiments, sequencing includes flowing a sequencing solution into one or more fluidic channels of the flow cell.

In an aspect is provided a method of sequencing a target polynucleotide in a sequencing device including a flow cell system as described herein. In embodiments, the method includes a) executing one or more sequencing cycles, each cycle comprising (i) flowing a sequencing solution through the fluidic channel and extending a complementary polynucleotide that is hybridized to an immobilized target polynucleotide, or completement thereof, by incorporating a first nucleotide using a polymerase; and (ii) detecting a label that identifies the first nucleotide; (b) extending the complementary polynucleotide in one or more dark cycles, wherein each dark cycle comprises flowing a dark solution through the fluidic channel and extending the complementary polynucleotide by one or more nucleotides using the polymerase, without performing a detection event to identify nucleotides incorporated during the dark cycle; and (c) executing one or more sequencing cycles, each cycle comprising (i) extending the complementary polynucleotide by incorporating a second nucleotide using a polymerase; and (ii) detecting a label that identifies the second nucleotide, thereby sequencing a target polynucleotide. By way of example, in another embodiment, a controlled dark cycle extension may be achieved by contacting template nucleic acid molecules with a pool of native nucleotides where one or more of the four nucleotide bases is absent. Here, the extension halts when the extending strand reaches a base on the template molecule (e.g., dA) whose complement is one of the absent bases (e.g., dT). In embodiments, prior to executing one or more sequencing cycles, the method includes flowing a clustering solution into one or more fluidic channels of the flow cell to generate clusters of immobilized target polynucleotides, or complements thereof.

In embodiments, the dark solution is a limited-extension solution. The limited-extension solution reaction mixture includes a plurality of nucleotides or analogs thereof wherein one, two, or three of the following nucleotide types are omitted from the dark solution: (a) adenine nucleotides and analogs thereof; (b) (i) thymine nucleotides and analogs thereof, and (ii) uracil nucleotides and analogs thereof (c) cytosine nucleotides and analogs thereof; or (iv) guanine nucleotides and analogs thereof. In embodiments, adenine nucleotides and analogs thereof are omitted. In embodiments, thymine nucleotides and analogs thereof, and uracil nucleotides and analogs thereof are omitted. In embodiments, cytosine nucleotides and analogs thereof are omitted. In embodiments, guanine nucleotides and analogs thereof are omitted.

In embodiments, the dark solution includes a plurality of adenine nucleotides, or analogs thereof thymine nucleotides, or analogs thereof, and cytosine nucleotides, or analogs thereof, and does not include a plurality of guanine nucleotides or analogs thereof. In embodiments, the dark solution includes a plurality of adenine nucleotides, or analogs thereof thymine nucleotides, or analogs thereof, and guanine nucleotides, or analogs thereof, and does not include a plurality of cytosine nucleotides or analogs thereof. In embodiments, the dark solution includes a plurality of adenine nucleotides, or analogs thereof guanine nucleotides, or analogs thereof, and cytosine nucleotides, or analogs thereof, and does not include a plurality of thymine nucleotides or analogs thereof. In embodiments, the dark solution includes a plurality of guanine nucleotides, or analogs thereof; thymine nucleotides, or analogs thereof, and cytosine nucleotides, or analogs thereof, and does not include a plurality of adenine nucleotides or analogs thereof. In embodiments, the limited-extension solution includes a plurality of adenine nucleotides, or analogs thereof; thymine nucleotides, or analogs thereof, and cytosine nucleotides, or analogs thereof, and does not include a plurality of guanine nucleotides or analogs thereof. In embodiments, the limited-extension solution includes a plurality of adenine nucleotides, or analogs thereof; thymine nucleotides, or analogs thereof, and guanine nucleotides, or analogs thereof, and does not include a plurality of cytosine nucleotides or analogs thereof. In embodiments, the limited-extension solution includes a plurality of adenine nucleotides, or analogs thereof; guanine nucleotides, or analogs thereof, and cytosine nucleotides, or analogs thereof, and does not include a plurality of thymine nucleotides or analogs thereof. In embodiments, the limited-extension solution includes a plurality of guanine nucleotides, or analogs thereof; thymine nucleotides, or analogs thereof, and cytosine nucleotides, or analogs thereof, and does not include a plurality of adenine nucleotides or analogs thereof. In embodiments, executing a sequencing cycle includes (i) incorporating in series with a nucleic acid polymerase, one of four differently labeled nucleotide analogues into a nucleic acid strand complementary to the template nucleic acid to create a sequenced-extension strand, where each of the four differently labeled nucleotide analogues include a detectable label; and (ii) detecting the unique detectable label of each incorporated nucleotide analogue, so as to thereby identify each incorporated nucleotide analogue in the sequenced-extension strand. Sequence data is collected for a first portion of the template nucleic acid under a first set of reaction conditions as the template nucleic acid is extended to generate an extension strand, for example by traditional sequence by synthesis (SBS) methodologies. Following a defined number of sequencing cycles (i.e., a series of nucleotide extension steps that are sequenced), the reaction conditions are changed to a second set of reaction conditions to initiate a limited-extension (LE) or dark cycle. The cycle is referred to as ‘dark’ since during this cycle, sequencing (i.e., nucleotide identification) is not taking place.

Each dark cycle includes extending the complementary polynucleotide by one or more nucleotides using the polymerase, without performing a detection event to identify nucleotides incorporated during the dark cycle. During a dark cycle, the extension strand from the nucleotide extension step completed during the sequencing cycle, referred to as the sequenced-extension strand, is elongated with nucleotides (e.g., native nucleotides) under a second set of reaction conditions. The extension strand generated during this limited-extension or dark cycle may be referred to as the dark-extension strand and is contiguous with the extension strand generated from the sequencing cycle. The identity of each nucleic acid incorporated into the nascent nucleic acid strand is not monitored during a dark or LE cycle. Any number of native nucleotides may be incorporated into the dark-extension strand until a nucleotide analogue having a polymerase-compatible cleavable moiety (i.e., a reversible terminator moiety) is incorporated, which temporarily halts the polymerase reaction until the moiety is removed. Once the moiety is removed, another sequencing cycle or an additional dark cycle may be initiated. In embodiments, a series of dark cycles are performed before changing the reaction conditions to perform a series of sequencing cycles.

In some embodiments, the dark cycle includes extending the complementary polynucleotide by at least two nucleotides using the polymerase; where at least one nucleotide does not include a reversible terminator, and at least one nucleotide includes a reversible terminator moiety and a label, and optionally performing a detection event to identify nucleotides incorporated during the dark cycle. This process would enable detecting the labeled nucleotide as a quality control measure, for example to check the synchronization of the process.

In other embodiments, the dark cycle includes extending the complementary polynucleotide by one or more nucleotides using a polymerase; where the extension is accomplished by a pool of native nucleotides lacking at least one of the four bases. For example, the dark cycle may include extending the complementary nucleotide in the presence of three nucleotides, e.g., dA, dG, and dC. The cycles of extension may continue until the complement of the missing nucleotide, e.g., dT, is necessary to continue extension

Sequencing includes, for example, detecting a sequence of signals within the particle. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the readout is accomplished by epifluorescence imaging. A variety of sequencing chemistries are available, non-limiting examples of which are described herein. In embodiments, sequencing includes extending a sequencing primer to incorporate a nucleotide containing a detectable label that indicates the identity of a nucleotide in the target polynucleotide, detecting the detectable label, and repeating the extending and detecting of steps. In embodiments, the methods include sequencing one or more bases of a template nucleic acid by extending a sequencing primer hybridized to a template nucleic acid (e.g., an amplification product of a target nucleic acid). In embodiments, the sequencing includes sequencing-by-synthesis, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

### Definitions

As used herein, the term “flow cell” or “flowcell” refers to the reaction vessel in a nucleic acid sequencing device. The flow cell is typically a glass slide containing one or more fluidic channels, through which fluidic solutions (e.g., polymerases, nucleotides, air, and buffers) may traverse. In embodiments, the flow cell includes 2 or more (e.g., 4) independent channels. The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® glass, silica, silica-based materials, carbon, metals, an optical fiber or optical fiber bundles, sapphire, or plastic materials such as COCs and epoxies. The particular material can be selected based on properties desired for a particular use. The flow cells used in the various embodiments can include millions of individual nucleic acid clusters, e.g., about 2-8 million clusters per channel. Each of such clusters can give read lengths of at least 25-100 bases for DNA sequencing. The systems and methods herein can generate over a gigabase (one billion bases) of sequence per sequencing experiment.

As used herein, the term “fluid” or “solution” or “reagent” may be used interchangeably and includes any liquid or gas. A fluid can include, for example, air, a sequencing reaction solution (such as aqueous buffer containing enzymes, salts, and nucleotides); a wash solution (an aqueous buffer); a cleave solution (an aqueous buffer containing a cleaving agent, such as a reducing agent such as Dithiothreitol (DTT), tris(2-carboxyethyl)phosphine) (TCEP), or Tris(3-hydroxypropyl)phosphine (THPP); or a cleaning solution (a dilute bleach, dilute NaOH, dilute HCl, deionized water). The fluid can be, for example, an aqueous solution which may contain buffers (e.g., saline-sodium citrate (SSC), tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA).

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers of the disclosure. In embodiments, the channel is filled by the one or more polymers, and flow through the channel (e.g., as in a sample fluid) is directed through the polymer in the channel. In embodiments, the channel contains a gel. The term “gel” in this context refers to a semi-rigid solid that is permeable to liquids and gases. Exemplary gels include, but are not limited to, those having a colloidal structure, such as agarose; polymer mesh structure, such as gelatin; or cross-linked polymer structure, such as polyacrylamide or a derivative thereof. Analytes, such as polynucleotides, can be attached to a gel or polymer material via covalent or non-covalent means. Exemplary methods and reactants for attaching nucleic acids to gels are described, for example, in US 2011/0059865 which is incorporated herein by reference. The analytes can be nucleic acids and the nucleic acids can be attached to the gel or polymer via their 3′ oxygen, 5′ oxygen, or at other locations along their length such as via a base moiety of the 3′ terminal nucleotide, a base moiety of the 5′ nucleotide, and/or one or more base moieties elsewhere in the molecule. In embodiments, the shape of the channel can include sides that are curved, linear, angled or a combination thereof. Other channel features can be linear, serpentine, rectangular, square, triangular, circular, oval, hyperbolic, or a combination thereof. The channels can have one or more branches or corners. The channels can connect two points on a substrate, one or both of which can be the edge of the substrate. The channels can be formed in the substrate material by any suitable method. For example, channels can be drilled, etched, or milled into the substrate material. Channels can be formed in the substrate material prior to bonding multiple layers together. Alternatively, or additionally, channels can be formed after bonding layers together. In an embodiment, at least one channel has a cross sectional shape of a circle, rectangle, oval, or any other shape. Preferably, the flow rates, fluid viscosities, compositions, and geometries and sizes of the channel are selected so that fluid flow is laminar. Guidance for making such design choices is readily available publicly available resources, for example Acheson, Elementary Fluid Dynamics (Clarendon Press, 1990), and from software for modeling fluidics systems, e.g. SolidWorks from Dassault Systems. In an embodiment, at least one channel has passage cross-sections in the range of tens of square microns to a few square millimeters (e.g., maximal cross-sectional dimensions of from about 500 μm to about 0.1 μm). In an embodiment, the flow rates in the range of from a few nL/sec to a hundreds of μL/sec. In an embodiment, volume capacities in are the range of from 1 μm to a few nL, e.g. 10-100 nL.

As used herein, the term “substrate” refers to a solid support material. The substrate can be non-porous or porous. The substrate can be rigid or flexible. A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor, silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. The substrate may include wells. The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, star shaped (with any number of vertices) etc. The cross section of a well taken orthogonally with the surface can be curved, square, polygonal, hyperbolic, conical, or angular. In embodiments, the substrate includes a plurality of wells, wherein the wells containing a polymer or gel material, and the wells are separated from each other by interstitial regions on the surface, the interstitial regions segregating the gel material in each of the wells from the gel material in other wells. In embodiments, a well is capable of including some volume of liquid. The minimum or maximum volume may be selected for enhancing desired characteristics, such as throughput, resolution, analyte composition, or analyte reactivity. For example, the volume can be at least 1×10⁻³ μm³, 1×10⁻² μm³, 0.1 μm³, 1 μm³, 10 μm³, 100 μm³ or more. In embodiments, the volume can be at most 1×10⁴ μm³, 1×10³ μm³, 100 μm³, 10 μm³, 1 μm³, 0.1 μm³ or less. It will be understood that a gel material or polymer can fill all or part of the volume of a well. The volume of gel in an individual well can be greater than, less than or between the values specified above. In embodiments, a gel layer can have a depth that is at least about 10 nm, 25 nm, 50 nm, 100 nm, 500 nm, 1 μm, 10 μm, 50 μm, 100 μm, 500 μm, 1 mm, 10 mm, 100 mm or higher. In embodiments, the depth of a gel layer can be at most about 100 mm, 10 mm, 1 mm, 500 μm, 100 μm, 50 μm, 10 μm, 1 μm, 500 nm, 100 nm, 50 nm, 25 nm, 10 nm or 1 nm. Wells may have a cross-sectional dimension of less than about 250 μm, less than about 100 μm, or less than about 50 μm. In some embodiments, wells can have a volume of less than 10 μL, less than 1 μL, less than 0.1 μL, less than 10 nL, less than 1 nL, less than 0.1 nL, or less than 10 pL.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art, and refers to a population of different molecules that are attached to one or more solid-phase substrates such that the different molecules can be differentiated from each other according to their relative location. A clow cell may include an array and can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example an array can have at least about 100 features/cm², at least about 1,000 features/cm², at least about 10,000 features/cm², at least about 100,000 features/cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher. Clustering refers to the process of generating clusters (i.e., solid-phase amplification of polynucleotides).

As used herein the terms “automated” and “semi-automated” mean that the operations are performed by system programming or configuration with little or no human interaction once the operations are initiated, or once processes including the operations are initiated.

It is to be understood that the phrase “fluidically connected” or “fluidly connected” may be used herein to describe connections between two or more components that place such components in fluidic communication with one another, much in the same manner that “electrically connected” may be used to describe an electrical connection between two or more components.

As used herein, the term “nucleic acid” refers to nucleotides (e.g., deoxyribonucleotides or ribonucleotides) and polymers thereof in either single-, double- or multiple-stranded form, or complements thereof. The terms “polynucleotide,” “oligonucleotide,” “oligo” or the like refer, in the usual and customary sense, to a sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA with linear or circular framework. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

As used herein, the term “polynucleotide template” refers to any polynucleotide molecule that may be bound by a polymerase and utilized as a template for nucleic acid synthesis. As used herein, the term “polynucleotide primer” refers to any polynucleotide molecule that may hybridize to a polynucleotide template, be bound by a polymerase, and be extended in a template-directed process for nucleic acid synthesis, such as in a PCR or sequencing reaction.

In general, the term “target polynucleotide” or “sample polynucleotide” refers to a nucleic acid molecule or polynucleotide in a starting population of nucleic acid molecules having a target sequence whose presence, amount, and/or nucleotide sequence, or changes in one or more of these, are desired to be determined. In general, the term “target sequence” refers to a nucleic acid sequence on a single strand of nucleic acid. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s).

As used herein, the term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence, only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence.

As used herein, the term “sequencing solution” or “sequencing reaction solution” is used in accordance with its plain and ordinary meaning and refers to an aqueous mixture that contains the reagents necessary to allow a nucleotide or nucleotide analogue to be added to a DNA strand by a DNA polymerase. In embodiments, a sequencing solution includes a plurality of modified nucleotides, salts, ions (e.g., Mg2⁺), buffers, and sequencing enzymes (e.g., a DNA polymerase). Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ξ DNA polymerase, Pol γ DNA polymerase, Pol θ DNA polymerase, Pol υ DNA polymerase, or a thermophilic nucleic acid polymerase (e.g. P. abyssi polymerase, Therminator γ, 9° N polymerase (exo-), Therminator II, Therminator III, or Therminator IX). In embodiments, the DNA polymerase is a modified archaeal DNA polymerase. In embodiments, the polymerase is a reverse transcriptase.

As used herein, a “native” nucleotide is used in accordance with its plain and ordinary meaning and refers to a naturally occurring nucleotide that does not include an exogenous label (e.g., a fluorescent dye, or other label) or chemical modification such as those that may characterize a nucleotide analog (e.g., a reversible terminating moiety). Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

As used herein, the term “modified nucleotide” refers to a nucleotide modified in some manner. Typically, a nucleotide contains a single 5-carbon sugar moiety, a single nitrogenous base moiety and 1 to three phosphate moieties. In embodiments, a nucleotide can include a blocking moiety (alternatively referred to herein as a reversible terminator moiety) and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

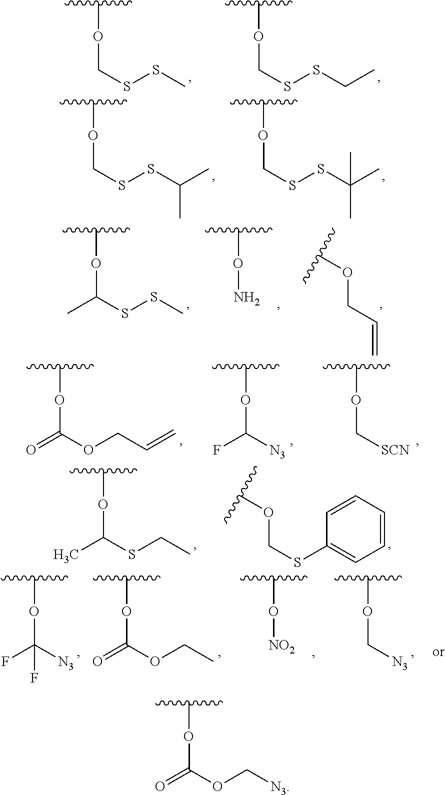

A label moiety of a nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein comprises contacting a template and an annealed primer with a suitable polymerase under conditions suitable for polymerase extension and/or sequencing. The sequencing methods are preferably carried out with the target polynucleotide arrayed on a solid substrate within a flow cell. In an embodiment, the sequencing is sequencing by synthesis (SBS). Briefly, SBS methods involve contacting target nucleic acids with one or more labeled nucleotides in the presence of a DNA polymerase. Optionally, the labeled nucleotides can further include a reversible termination property that terminates extension once the nucleotide has been incorporated. Thus, for embodiments that use reversible termination, a cleaving solution can be delivered to the flow cell (before or after detection occurs). Washes can be carried out between the various delivery steps. The cycle can then be repeated n times to extend the primer by n nucleotides, thereby detecting a sequence of length n. Exemplary SBS procedures and detection platforms that can be readily adapted for use with the methods of the present disclosure are described, for example, in Bentley et al., Nature 456:53-59 (2008), WO 2004/018497; and WO 2007/123744, each of which is incorporated herein by reference in its entirety. In an embodiment, sequencing is pH-based DNA sequencing. The concept of pH-based DNA sequencing, has been described in the literature, including the following references that are incorporated by reference: US2009/0026082; and Pourmand et al, Proc. Natl. Acad. Sci., 103: 6466-6470 (2006) which are incorporated herein by reference in their entirety. Other sequencing procedures that use cyclic reactions can be used, such as pyrosequencing. Sequencing-by-ligation reactions are also useful including, for example, those described in Shendure et al. Science 309:1728-1732 (2005). Sequencing can include a plurality of sequencing cycles.

As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the complementary polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes and other substances can be removed between steps by washing. Cycles may include repeating these steps, and the sequence of each cluster is read over the multiple repetitions.

A nucleic acid can be amplified by a suitable method. The term “amplified” and “amplification” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof. In some embodiments an amplification reaction comprises a suitable thermal stable polymerase. Thermal stable polymerases are known in the art and are stable for prolonged periods of time, at temperature greater than 80° C. when compared to common polymerases found in most mammals. In certain embodiments the term “amplified” refers to a method that comprises a polymerase chain reaction (PCR). Conditions conducive to amplification (i.e., amplification conditions) are well known and often comprise at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support. In embodiments, the device includes a heating element capable of heating the fluids and/or the flow cell. The heating element may be a resistive heater, inductive heater, peltier/thermoelectric, or radiative heater (e.g., infrared heater). The heating element may be comprised of any suitable material. For example, the heating element may include metals, such as nichrome, kanthal, cupronickel, and the like. In embodiments, the heating element includes a ceramic material (e.g., molybdenum disilicide, silicon carbine, barium titanate, lead titanate, or quartz). The heating element may include PTC rubber (i.e., polydimethylsiloxane (PDMS) loaded with carbon nanoparticles). The heating element may be a resistive heater comprised of any suitable material. The heating element may include an etched resistive metal film (e.g., an etched nichrome resistive metal film). The heating element may include a resistance heating alloy wire. The heating element may include additional insulating elements. The heating element may include an etched nichrome resistive metal film with Kapton insulation. In embodiments, the heating element is a heated tube. The tube may be rigid (i.e., fixed) or flexible. In embodiments, a wire is wrapped on the tube and then it is covered with insulation material (e.g., Kapton, polymer, steel wire or silicone). In embodiments, the heating element is a nickel inductive heater. A heating element that includes nickel may be selected as the induction heating element in the microfluidic device because of the relatively small influence of geometries and faster thermal response. A heating element provides heat (e.g., an increase in temperature).

In some embodiments solid phase amplification comprises a nucleic acid amplification reaction comprising only one species of oligonucleotide primer immobilized to a surface or substrate. In certain embodiments solid phase amplification comprises a plurality of different immobilized oligonucleotide primer species. In some embodiments solid phase amplification may comprise a nucleic acid amplification reaction comprising one species of oligonucleotide primer immobilized on a solid surface and a second different oligonucleotide primer species in solution. Multiple different species of immobilized or solution based primers can be used. Non-limiting examples of solid phase nucleic acid amplification reactions include interfacial amplification, bridge amplification, emulsion PCR, WildFire amplification (e.g., US patent publication US 2013/0012399 (incorporated by reference), the like or combinations thereof.

As used herein, the term “extending,” “extension,” or “elongation” is used in accordance with their plain and ordinary meanings and refer to synthesis by a polymerase of a new polynucleotide strand complementary to a template strand by adding free nucleotides from a reaction mixture that are complementary to the template in a 5′-to-3′ direction, including condensing a 5′-phosphate group of a dNTPs with a 3′-hydroxy group at the end of the nascent (elongating) DNA strand.

The term “cross-contamination” in the context of two independent reactions refers to substances from the first reaction that are present in detectable quantities in the second reaction. Cross-contamination does not include substances which are common to the first and second reactions (e.g., water, buffers, salts, or enzymes). In aspects and embodiments described herein, the methods and systems achieve minimal cross-contamination. In embodiments, minimal cross-contamination is when a substance from the first reaction is not detected in the second reaction. In embodiments, minimal cross-contamination is when a substance from the first reaction is not detected in the second reaction within the limits of detection for the substance. In embodiments, minimal cross-contamination is when a substance from the first reaction solution is detected in a quantity less than 0.01%, 0.02%, 0.03%, 0.04%, or 0.05% of the volume in the second reaction solution. In embodiments, minimal cross-contamination is when a substance from the first reaction solution is detected in a quantity less than 0.1%, 0.2%, 0.3%, 0.4%, or 0.5% of the volume in the second reaction solution.

The term “nucleic acid sequencing device” means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems (e.g., one or more lasers and one or more optical sensors such as a camera, objective, and lenses for detecting fluorescence), data collection and/or integration systems, for the purpose of determining the nucleic acid sequence of a template polynucleotide. Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the device includes an imaging system. The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a fluorescence spectrophotometer including an objective lens and/or a solid-state imaging device. The solid-state imaging device may include a charge coupled device (CCD) and/or a complementary metal oxide semiconductor (CMOS).

As used herein, the terms “label” and “labels” are used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF dyes (Biotium, Inc.), Alexa Fluor dyes (Thermo Fisher), DyLight dyes (Thermo Fisher), Cy dyes (GE Healthscience), IRDyes (Li-Cor Biosciences, Inc.), and HiLyte dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide comprises a label (such as a dye). In embodiments, the label is not associated with any particular nucleotide, but detection of the label identifies whether one or more nucleotides having a known identity were added during an extension step (such as in the case of pyrosequencing).

“Contacting” is used in accordance with its plain ordinary meaning and refers to the process of allowing at least two distinct species (e.g., chemical compounds including biomolecules, particles, solid supports, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In embodiments contacting, includes allowing a sample as described herein to interact with a flow cell.

All patents, patent applications, articles and publications mentioned herein, both supra and infra, are hereby expressly incorporated herein by reference in their entireties.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” means a range of values including the specified value, which a person of ordinary skill in the art would consider reasonably similar to the specified value. In embodiments, the term “about” means within a standard deviation using measurements generally acceptable in the art. In embodiments, about means a range extending to +/−10% of the specified value. In embodiments, about means the specified value.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flow(s) depicted in the accompanying figures and/or described herein do not necessarily require the particular order shown, or sequential order, to achieve desirable results. Other implementations may be within the scope of the following claims.

1. A flow cell system, comprising:
at least one flow cell configured to serve as a reaction vessel in a nucleic acid sequencing device, the flow cell including at least one fluidic channel through which a fluid solution can flow;
an inlet to the at least one fluidic channel;
an outlet to the at least one fluidic channel;
wherein two or more independent reactions can occur on the at least one flow cell with minimal or a reduction in cross-contamination.

2. The flow cell system of claim 1, wherein the at least one fluidic channel includes a first fluidic channel and a separate, second fluidic channel.

3. The flow cell system of claim 1, wherein the inlet provides a structure wherein a first fluid can both exit and enter a first fluidic channel.

4. The flow cell system of claim 3, wherein the outlet provides a structure wherein a first fluid can both exit and enter a second fluidic channel.

5. The flow cell system of claim 1, wherein the wherein the at least one fluidic channel includes a first fluidic channel that serves as a first bi-directional channel, and a separate, second fluidic channel that serves as a second bi-directional channel, and wherein the inlet serves as structure where a first fluid can both exit and enter the first bi-directional channel, and the outlet serves as a structure where a second fluid can both exit and enter the second bi-directional channel.

6. The flow cell system of claim 1, wherein the at least one fluidic channel includes one, two, three, four, or more fluidic channels.

7. The flow cell system of claim 1, further comprising a sequencing manifold fluidly coupled to the at least one fluid channel.

8. The flow cell system of claim 7, further comprising a clustering manifold fluidly coupled to the at least one fluid channel.

9. The flow cell system of claim 8, further comprising a cleaving solution reservoir fluidly coupled to the at least one flow cell.

10. The flow cell system of claim 8, further comprising a waste reservoir and sample cartridge fluidly coupled to the at least one flow cell.

11. The flow cell system of claim 1, further comprising at least one pressure source configured to drive fluid through the at least one fluidic channel.

12. The flow cell system of claim 1, wherein the fluid solution includes at least one of a sequencing solution and a clustering solution.

13. The flow cell system of claim 8, wherein the sequencing solution is delivered into the flow cell from one side of the flow cell, and the clustering solution is delivered into the flow cell from an opposite side of the flow cell.

14. The flow cell system of claim 1, wherein the at least one flow cell includes a plurality of flow cells.

15. The flow cell system of claim 1, wherein the at least one flow cell includes two or more flow cells.

16. The flow cell system of claim 1, wherein the at least one flow cell includes 2 or 4 flow cells, wherein each flow cell comprises four fluidic channels.

17. The flow cell system of claim 1, wherein the at least one fluidic channel includes four fluidic channels.

18. A method of performing a reaction on a flow cell, comprising:
performing at least two independent reactions on a common flow cell with minimal or reduced cross-contamination.

19. The method of claim 18, wherein the flow cell is configured to serve as a reaction vessel in a nucleic acid sequencing device, and wherein the flow cell comprises:
at least one fluidic channel through which a fluid solution can flow;
an inlet to the at least one fluidic channel; and
an outlet to the at least one fluidic channel.

20. The method of claim 18, wherein the two independent reactions include a sequencing reaction and an amplification reaction.

21. The method of claim 18, wherein the at least two independent reactions include a first reaction and a second reaction, and wherein the first reaction and the second reaction in occur within a common flow channel of the flow cell.

22. The method of claim 21, wherein the first reaction and the second reaction occur at a common time or at a different time.

23. The method of claim 18, wherein the at least two independent reactions include a first reaction and a second reaction, and wherein the first reaction occurs within a first flow channel of the flow cell and the second reaction occurs within a second flow channel of the flow cell.

24. A method of amplifying and sequencing a target polynucleotide in a sequencing device comprising a flow cell system of claim 1, said method comprising:
a) contacting the flow cell with a target polynucleotide and amplifying the target polynucleotide to generate a plurality of immobilized template nucleic acids, wherein each immobilized template nucleic acid comprises the target polynucleotide or a complement thereof; and
b) sequencing the plurality of immobilized template nucleic acids;
thereby amplifying and sequencing a target polynucleotide in a sequencing device.

25. A method of sequencing a target polynucleotide in a sequencing device comprising a flow cell system of claim 1, said method comprising:
a) executing one or more sequencing cycles, each cycle comprising (i) flowing a sequencing solution through the fluidic channel and extending a complementary polynucleotide that is hybridized to the template nucleic acid by incorporating a first nucleotide using a polymerase; and (ii) detecting a label that identifies the first nucleotide;
(b) extending the complementary polynucleotide in one or more dark cycles, wherein each dark cycle comprises flowing a dark solution through the fluidic channel and extending the complementary polynucleotide by one or more nucleotides using the polymerase, without performing a detection event to identify nucleotides incorporated during the dark cycle; and
(c) executing one or more sequencing cycles, each cycle comprising (i) extending the complementary polynucleotide by incorporating a second nucleotide using a polymerase; and (ii) detecting a label that identifies the second nucleotide, thereby sequencing a target polynucleotide.
